(19) United States
(12) Patent Application Publication
(10) Pub. No.: US 2009/0215141 A1
(43) Pub. Date: Aug. 27, 2009

(54) BIOFUEL GENERATING SYSTEM

(76) Inventor: David TESSEL, Riverdale, NY (US)

Correspondence Address: OSTROLENK FABER GERB & SOFFEN, 1180 AVENUE OF THE AMERICAS, NEW YORK, NY, 100368403, US

(21) Appl. No.: 12/390,891

(22) Filed: Feb. 23, 2009

Related U.S. Application Data
(60) Provisional application No. 61/030,716, filed on Feb. 22, 2008.

(51) Int. Cl. C12P 5/02; C12M 1/00

(52) U.S. Cl. 435/167; 435/300.1

(57) ABSTRACT

A biofuel generating method and biofuel generating device comprising operationally linking an anaerobic digester with a hydroponic biomass energy crop production system in a symbiotic relationship. The hydroponic system continually provides high energy containing biomass crops as feed stock to the anaerobic digestor and the anaerobic digestor provides nitrogenous digestate into the nutrient hydroponic system as a nutrient, to facilitate growth of additional biomass feed stock. The anaerobic digestor further provides the requisite carbon dioxide for the hydroponic crop feed stock to stimulate germination of crop seeds and the growth of crop seedlings. Heat generated from the anaerobic digestion process is also optionally channeled to the hydroponic system to further accelerate and promote hydroponic feed stock crop growth. The anaerobic digester produces combustible fuel including methane gas during the digestion process.

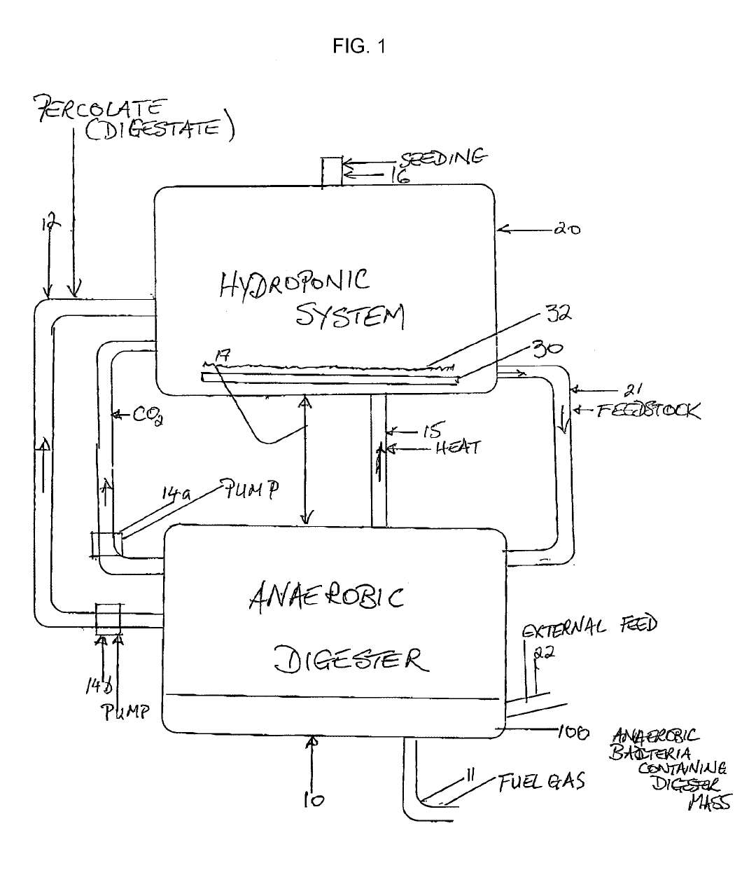

This application is based on and claims priority to U.S. Provisional Patent Application Ser. No. 61/030,716, filed on Feb. 22, 2008 and titled BIOFUEL GENERATING SYSTEM INCLUDING AN ANAEROBIC SYSTEM WITH AN INTEGRATED HYDROPONIC CROP GROWING SYSTEM, the entire contents of which are hereby incorporated by reference.

### FIELD OF THE INVENTION

The present invention relates to the generation of fuel with biofuel feed stock and particularly to the generation of fuel gas with the use of anaerobic digesters.

### BACKGROUND OF THE INVENTION

Anaerobic digestion is a series of processes in which microorganisms such as bacteria break down biodegradable material in the absence of oxygen. It is widely used to treat wastewater sludges and organic wastes because it provides volume and mass reduction of the input material. As part of an integrated waste management system, anaerobic digestion reduces the emission of landfill gas into the atmosphere. Anaerobic digestion is a renewable energy source because the process produces a methane and carbon dioxide rich biogas suitable for energy production, helping replace fossil fuels. Also, the nutrient-rich solids left after digestion can be used as fertilizer. Anaerobic digesters in their simplest form are containers or contained areas with the anaerobic digesting microorganisms into which biomass feedstock is provided and from which methane and carbon dioxide are generated in addition to the digestate or percolate, with nutrients.

In order to increase fuel yield, since wastewater sludges and organic wastes often have been stripped of a substantial part of energy content and where fuel generation is the primary objective, energy crops are often cultivated and harvested for use as the biomass feed stock for the anaerobic digesters. The most common energy crops are trees and perennial grasses which can often be grown on farm land that is less suitable for conventional crops. Grasses are particularly utilized where short growing cycles are required. Economic factors also play an important part in rendering anaerobic digesters suitable for energy production in the face of more profitable food crops and the high costs involved in transporting and handling wastewater sludges and organic wastes. Anaerobic digester systems are known and are described in prior patents as in recent U.S. Pat. No. 7,481,940 and in older U.S. Pat. Nos. 4,100,023 and 4,329,428, the full contents and disclosures of which are incorporated herein in their entireties by reference.

Hydroponic growth systems, i.e., soilless crop growth in mineral nutrient solutions have been extensively utilized almost primarily for the cultivation of food crops, with hydroponic growth systems providing much higher crop yields and being feasible where ordinary agriculture or gardening is impossible. Hydroponics and hydroponic systems are known and are described in prior patents such as in container based systems described in U.S. Pat. Nos. 3,992,809; 3,991,514; and 3,945,148, the entire contents and disclosures of which are incorporated herein in their entireties by reference.

In general terms, hydroponics is a technology for growing plants in nutrient solutions (water and fertilizers) with or without the use of artificial medium (e.g., sand, gravel, vermiculite, rockwool, peat, coir, sawdust) to provide mechanical support. Liquid hydroponic systems have no other supporting medium for the plant roots: aggregate systems have a solid medium of support. Hydroponic systems are further categorized as “open”, where after the nutrient solution has been delivered to the plant roots, it is not reused; or “closed” where surplus solution is recovered, replenished, and recycled. All commercial hydroponic systems in temperate regions of the world are enclosed in greenhouse-type structures to provide temperature control, reduce evaporative water loss, and to reduce disease and pest infestations.

### SUMMARY OF THE INVENTION

It is accordingly an object of the present invention to provide an efficient and economical method of generating biofuel and a biofuel generating system with high yield and minimal operating costs, with the use of an anaerobic digestor having an integrated biomass feed stock source.

It is a further object of the present invention to directly link an anaerobic digester with a hydroponic system in an interdependent biofuel generating system wherein each provides operational requirements for the other, in a symbiotic, efficient, low cost operational cycle, requiring minimal maintenance and ground space.

Generally the present invention comprises a biofuel generating method and biofuel generating device comprising operationally linking an anaerobic digester with a hydroponic biomass energy crop production system in a symbiotic relationship wherein the hydroponic system continually provides high energy containing biomass crops as feed stock to the anaerobic digestor and wherein the anaerobic digestor provides nitrogenous percolate or digestate into the nutrient hydroponic system as a nutrient, to facilitate growth of additional biomass feed stock. The anaerobic digestor further provides the requisite carbon dioxide for the hydroponic crop feed stock to stimulate germination of crop seeds and the growth of crop seedlings. Heat generated from the anaerobic digestion process is also optionally channeled to the hydroponic system to further accelerate and promote hydroponic feed stock crop growth. The anaerobic digester produces combustible fuel including methane gas during the digestion process. This fuel is periodically or continuously harvested from the anaerobic digester. To complete the cycling operation, the hydroponic system biomass crops are replenished with periodic reseeding. The use of pumping elements and gravity feed, as appropriate, facilitates the symbiotic cycling process between the anaerobic digester and the hydroponic system. Initiation of use of the device for fuel generation requires seeding of the hydroponic system with seeds for growing the biomass feed stock crop before anaerobic digester contribution to the symbiotic system. In the initial stages the hydroponic system is provided with nutrients and carbon dioxide from external sources. Alternatively, the anaerobic digester is initially primed with externally provided digestable biomass. The respective materials are transferred via conduits which connect the anaerobic digester and hydroponic system

The above and other objects, features and advantages of the present invention will become more evident from the following disclosure and drawings in which:

### SHORT DESCRIPTION OF THE DRAWING

The sole FIGURE is a schematic representation of the integrated anaerobic digester and hydroponic system of the present invention as a single basic unit.

### DETAILED DESCRIPTION OF THE DRAWING AND PREFERRED EMBODIMENTS

In accordance with the present invention, and as shown in the FIGURE, an anaerobic digester 10 is directly linked to a hydroponic crop growing system 20 with respective intakes and outlets. For economy and efficiency, the digester and hydroponic system are preferably stacked within a single space-saving footprint, with a substantial saving in terms of land use. As shown, the digester 10 is physically positioned below the hydroponic system 20 This arrangement can be reversed or multiple digesters or hydroponic systems may be linked to form an energy generating systems in a relatively small footprint tower. At least one digester and one hydroponic system constitute a unit of the biofuel generator of the present invention. The biofuel generating unit is however not limited to a one-to-one ratio and one digester may be linked to and used with multiple hydroponic systems and one hydroponic system may be linked to and used with multiple digesters.

A factor in determining the relative size or number of digesters and hydroponic systems and ratios is the nature of the energy crop being grown and its growth rate. It is desirable that the growth rate and crop harvest yield be matched to the digestion rate to minimize idle time for either the digester(s) or the hydroponic system(s) and to maximize biofuel production.

The hydroponic system is preferably used to grow high energy yielding crops such as barley grass which has a maturation rate to harvesting of about 14 days. Variations in growth rate are also dependent on factors such as crop and fertilizer type and amount, as well the amount of heat and light provided and generally a number of other art recognized other requirements for hydroponic growth.

Digestion residence times generally range from about 14 days to about 40 days in single stage thermophilic digesters and two-stage mesophilic digesters, depending on the particular anaerobic systems being utilized. It is understood that efficiencies can be effected with the controlled feed stock systems of the present invention which requires little or no separation of materials and minimal odor treatment, as is usually required with treatment of wastewater sludges and organic waste. In addition, the controlled high energy yielding crops maximize the yield of the methane fuel being emitted and harvested.

It is common in anaerobic digester systems used in digesting waste to require separation and removal of non-digestible materials in order to maximize the digestion process relative to area being used. It is accordingly desirable in the operation of the present invention to further minimize separation operations, by using trays made of anaerobically-digestible organic matter, coated with organic vegetable oils to render them impervious to water in the hydroponic system. As a result, no separation of growing system trays is necessary either before or after the anaerobic digestion processes and the biomass crops are provided to the anaerobic digester together with their supporting trays. The trays are then replaced with the periodic reseeding of the hydroponic system.

The fuel generating system shown in the FIGURE schematically depicts for simplicity a minimal unit of a single hydroponic system 20 operationally interconnected with a single anaerobic digester 10. Crop seeds such as of barley grass are initially directly placed in the hydroponic system 20 and replenished via input 16. Alternatively removal of an upper removable cover 21 permits direct placement of crop seed and replacement of trays 30 as support for the seeds and crops.

The hydroponic system 20 is primed initially with solution fertilizer or nutrients, as is common in hydroponic systems, in a carbon dioxide containing atmosphere. The hydroponic system is heated or kept warm to accelerate biomass crop growth. Thereafter, the crop is harvested, when sufficiently grown, by a gravity drop into anaerobic digester 10 via chute 21 into anaerobic bacteria containing bed 100, where it becomes the feed stock for the digester. To facilitate this feed stock movement, tilting mechanism 17, such as an hydraulic lift, raises one side of the hydroponic system 20 opposite the chute 21 to effect a gravity drop through chute 21 of tray 30, with biomass crop 32 thereon. Mechanism 17 is thereafter returned to a level placement of the hydroponic system. Alternatively, a conveyor system (not shown) may be provided to transport the biomass crop to the digester, to minimize handling of the biomass.

While anaerobic digestion is taking place, the hydroponic system is reseeded and primed as before. After and during digestion, generated methane fuel gas is harvested via fuel outlet 11 and generated carbon dioxide is cycled via outlet 13 to the hydroponic system 20 with pump 14a, to facilitate new biomass crop growth. Heat from the exothermic digestion process is cycled to the hydroponic system 20 via conduit 15 and liquid digestate or percolate is pumped by pump 14b to the hydroponic system 20 as nutrient for new crop growth. Optionally, if there is a mismatch between crop growth rate and digestion time with growth rate time exceeding digestion time, additional feed stock may be added to the anaerobic digester via inlet 22 to maintain efficient use of the digester.

In the embodiment shown, the hydroponic system 20 is above the digester 10. The relative position may be reversed or they may be positioned laterally adjacent each other with appropriate deployment of pumping elements. It is also possible to provide multiple hydroponic system elements with digester units (e.g., above and below the digester, or in multiple stacks with a common methane gas collector) depending on land use footprint availability or by taking into account efficiencies in matching growth and digestion rates. Fuel output requirements, such as for dedicated powering of facilities, including production plants, multiple residences or office facilities, may also dictate the configuration and number of hydroponic system/anaerobic digester units and amount of biofuel generated.

It is understood that the above description and drawing are merely exemplary of the present invention and that changes in components and component constituents and procedures are possible without departing from the scope of the present invention as defined in the following claims.

What is claimed is:

1. A method for the generation of biofuel comprising the steps of:
operatively connecting a biofuel producing anaerobic digester with a hydroponic biomass crop producing system in a biofuel producing unit, the hydroponic biomass crop producing system configured to continually provide biomass fuel stock crop to the anaerobic digestor and the anaerobic digestor being configured to anaerobically digest the biomass crop and provide nutrients and carbon dioxide generated during the digestion to an additional biomass crop growing in the hydroponic system, and to produce biofuel gas.

2. The method of claim 1, including:
a) seeding the hydroponic biomass producing system with seeds for the biomass crop;
b) allowing the seeds to germinate and grow into the biomass crop;
c) feeding the biomass crop to the anaerobic digester as feed stock;
d) allowing the anaerobic digester to digest the biomass crop and generate carbon dioxide, biofuel gas and nutrient containing digestate;
e) re-seeding the hydroponic biomass producing system with seeds for a new biomass crop
f) introducing nutrient digestate and carbon dioxide, generated by the anaerobic digester, to the reseeded hydroponic system;
g) harvesting the biofuel gas from the anaerobic digester;
h) repeating steps c through g.

3. The method of claim 1, wherein the anaerobic digester and hydroponic system are vertically stacked.

4. The method of claim 3, wherein biofuel gas producing units, each comprising at least one anaerobic digester and at least one hydroponic system, are vertically stacked and interconnected to produce biofuel gas.

5. The method of claim 3, wherein the hydroponic system is situated above the anaerobic digestor and wherein the hydroponic system is tilted to cause the biomass crop therein to slide into the anaerobic digester with a gravity feed.

6. The method of claim 1, wherein heat is generated in the anaerobic digester in an exothermic reaction during digestion of the biomass crop and wherein the generated heat is channeled to the hydroponic system.

7. The method of claim 1, wherein biomass crop growth rate is substantially matched to rate of digestion.

8. The method of claim 7, wherein the biomass crop is comprised of perennial grass.

9. The method of claim 8, wherein the biomass crop is comprised of barley grass.

10. The method of claim 3, wherein organically digestable and water resistant trays are used as supports for the biomass crop, with the trays being fed with the biomass crop into the anaerobic digestor and digested together with the biomass crop and wherein the trays are replaced in the hydroponic system prior to the step of subsequent seeding.

11. The method of claim 6, wherein any or all of the generated digestate, carbon dioxide and heat are pumped from the anaerobic digester to the hydroponic system.

12. A device for generating biofuel gas, the device comprising:
a) at least one unit comprised of at least one hydroponic system, and
b) at least one anaerobic digester,
the at least one hydroponic system and the at least one anaerobic digester being interconnected with respective conduits through which biomass crops grown in the at least one hydroponic system are fed as feedstock to the at least one anaerobic digester and wherein carbon dioxide gas and nutrient containing digestate, generated by the at least one anaerobic digestor, are provided through other respective conduits to the at least one hydroponic system and wherein the at least one anaerobic digestor has an external outlet for the collection of biofuel gas generated by the at least one anaerobic digester.

13. The device of claim 12, wherein at least one hydroponic system and at least one anaerobic digestor of the at least one unit are vertically stacked.

14. The device of claim 12, wherein the at least one anaerobic digester and the at least one hydroponic system are connected with at least one respective conduit through which heat generated by the at least one anaerobic digester is provided to the at least one hydroponic system.

15. The device of claim 13, wherein at least one hydroponic system is stacked above at least one anaerobic digester with the device further including at least one respective member which tilts the at least one hydroponic system to cause the biomass crop in the at least one hydroponic system to flow by gravity feed to the at least one anaerobic digester.

16. The device of claim 12, wherein the device is provided with at least one pump to accelerate the flow of carbon dioxide and digestate from the at least one anaerobic digester to the at least one hydroponic system.

17. The device of claim 12, wherein organically digestable and water resistant trays are used as supports for the biomass crop in the at least one hydroponic system, with the trays being adapted to be fed with the biomass crop into the at least one anaerobic digester and digested therein.
